(19) United States
(12) Patent Application Publication
(10) Pub. No.: US 2021/0228765 A1
(43) Pub. Date: Jul. 29, 2021

(54) SELF-ACTIVATING CATHETER INSERTION SITE DRESSING

(71) Applicant: Becton, Dickinson and Company, Franklin Lakes, NJ (US)

(72) Inventors: Gidon Ofek, Millcreek, UT (US); Brendan Laine, Salt Lake City, UT (US); Christopher Quach, Salt Lake City, UT (US)

(21) Appl. No.: 17/143,919

(22) Filed: Jan. 7, 2021

Related U.S. Application Data
(60) Provisional application No. 62/966,847, filed on Jan. 28, 2020.

(51) Int. Cl. A61L 15/56; A61L 15/22; A61L 15/42; A61L 15/28; A61L 15/32; A61M 25/02

(52) U.S. Cl. CPC A61L 15/56; A61L 15/225; A61L 15/425; A61L 15/28; A61L 15/325; A61M 2025/0273; A61L 2300/114; A61L 2300/404; A61L 2300/206; A61L 2300/104; A61M 25/02

(57) ABSTRACT

A self-activating dressing for use with a medical device inserted into a skin surface of a patient via a skin insertion site. The dressing includes a dressing body impregnated with a nitric oxide releasing compound which reacts in the presence of a physiological fluid to release nitric oxide. Nitric oxide provides antimicrobial activity and promotes wound healing. The nitric oxide releasing compound may include s-nitroso-n-acetyl penicillamine (SNAP), s-nitrosoglutathione (GSNO), and mixtures thereof. A slit defined in the dressing body enables the dressing body to be placed around a perimeter of the medical device on the skin surface at the skin insertion site such that the dressing body surrounds and contacts skin insertion site. The dressing body may be further impregnated with a catalyst, such as copper, iron, zinc, selenium, and silver, to facilitate release of nitric oxide. The dressing body may be further impregnated with an additional antimicrobial agent.

### RELATED APPLICATION

This application claims the benefit of U.S. Provisional Application No. 62/966,847, filed Jan. 28, 2020, and entitled SELF-ACTIVATING CATHETER INSERTION SITE DRESSING, which is incorporated herein in its entirety.

### BACKGROUND

The present disclosure relates to a catheter insertion site dressing impregnated with a nitric oxide pre-cursor that is activated in the presence of physiological fluids to release nitric oxide. Nitric oxide provides antimicrobial activity and promotes wound healing.

Catheters are commonly used for a variety of infusion therapies. Infusion therapy is one of the most common health care procedures. Hospitalized, home care, and other patients receive fluids, pharmaceuticals, and blood products via a vascular access device inserted into the vascular system. Infusion therapy may be used to treat an infection, provide anesthesia or analgesia, provide nutritional support, treat cancerous growths, maintain blood pressure and heart rhythm, or many other clinically significant uses. For example, catheters are used for infusing fluids, such as normal saline solution, various medicaments, and total parenteral nutrition into a patient, withdrawing blood from a patient, as well as monitoring various parameters of the patient's vascular system.

Catheters are commonly introduced into the vasculature of a patient as part of an intravenous catheter assembly. The catheter assembly generally includes a catheter hub, which supports the catheter, the catheter hub being coupled to a needle hub which supports an introducer needle. The introducer needle is extended and positioned within the catheter such that a beveled portion of the needle is exposed beyond a tip of the catheter. The beveled portion of the needle is used to pierce the skin of the patient to provide an opening whereby to insert the needle in the vasculature of the patient. Following insertion and placement of the catheter, the introducer needle is removed from the catheter thereby providing intravenous access to the patient.

Catheter use causes a breach of the skin that provides an access point for pathogens to enter the body, placing the patient at risk for local and systemic infectious complications. The potential for infection may be increased by proliferation of bacteria within or underneath a dressing at the catheter insertion site. Skin flora is the main source of microbial contamination and is responsible for approximately 65% of catheter related infections. Bacteria from the skin migrate along the external surface of the catheter and colonize the intravascular catheter tip leading to catheter related blood stream infections. Catheter-related bloodstream infection (CRBSI) is the third most common health care-acquired infection in the United States and is considered one of the most dangerous complications for patients. These infections are an important cause of illness and excess medical costs, as approximately 250,000-400,000 cases of central venous catheter (CVC) associated bloodstream infections occur annually in United States hospitals. In addition to the monetary costs, these infections are associated with anywhere from 20,000 to 100,000 deaths each year. Despite guidelines to help reduce healthcare associated infections (HAIs), catheter-related bloodstream infections continue to plague our healthcare system. Most organisms responsible for CRBSIs originate from the insertion site of the catheter; therefore, decreasing bacterial colonization at the insertion site may help reduce the incidence of CRBSIs.

While antimicrobial dressings for use with catheters and other percutaneous medical devices at the insertion site are known, there remains a need for a catheter insertion site dressing that is self-activating to provide broad-spectrum antimicrobial activity and promote wound healing at the insertion site.

The subject matter disclosed and claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to a self-activating dressing for use with a medical device inserted into a skin surface of a patient at a skin insertion site. The dressing body is impregnated with a nitric oxide releasing compound which reacts in the presence of a physiological fluid to release nitric oxide and provide antimicrobial activity and wound healing. The dressing body contains a slit configured to enable the dressing body to be placed around a perimeter of the medical device on the skin surface at the skin insertion site such that the dressing body surrounds and contacts skin insertion site.

Nitric oxide is an effective broad-spectrum antimicrobial and homeostasis agent for preventive and therapeutic applications. Nitric oxide released from an insertion site dressing promotes healing and antimicrobial protection. In addition, nitric oxide possesses synergistic properties with common antimicrobial agents, like chlorhexidine or silver, for enhanced functionality. One or more nitric oxide releasing compounds are integrated into the self-activating antimicrobial insertion site dressing, which releases nitric oxide in the presence of a physiological fluid. Non-limiting examples of a physiological fluid include sweat, interstitial fluid, and blood.

Any physiologically compatible nitric oxide releasing compound may be used herein. Non-limiting examples of nitric oxide releasing compounds include s-nitroso-n-acetylpenicillamine (SNAP), s-nitrosoglutathione (GSNO), and mixtures thereof. The nitric oxide releasing compound is impregnated in the dressing body.

The impregnating step may be accomplished by exposing the dressing body to a solvent having the nitric oxide releasing compound dissolved therein. The dressing body is exposed to the solvent solution for sufficient time to permit the nitric oxide releasing compound to penetrate the dressing body. The impregnating step may occur at room temperature. The impregnating step may occur at a temperature in the range from about 25 to 55° C. Any solvent that is compatible with the nitric oxide releasing compound and dressing body may be used. The nitric oxide releasing compound may be dissolved in tetrahydrofuran (THF), dioxolane, methyl ethyl ketone (MEK), methanol, ethanol, isopropyl alcohol, water, or combinations thereof. The dressing may be soaked in these solutions containing the nitric oxide releasing compound for sufficient time to impregnate the dressing with the nitric oxide releasing compound. The exposure time may range between 5 minutes and 24 hours.

The dressing body may be further impregnated with a catalyst to facilitate release of nitric oxide. Non-limiting examples of such catalysts include copper, iron, zinc, selenium, and silver. The catalyst may be impregnated into the dressing body by exposing the dressing body to a solvent having the catalyst dissolved therein. The catalyst may be impregnated into the dressing body using the same solvent system as the nitric oxide releasing compound, discussed above, either during the same impregnation step, a subsequent impregnation step, or a prior impregnation step. The dressing body is exposed to the solvent solution for sufficient time to permit the catalyst to penetrate the dressing body. The impregnating step may occur at room temperature. The impregnating step may occur at a temperature in the range from about 25 to 55° C. Any solvent that is compatible with the catalyst and dressing body may be used, including those described above in relation to the nitric oxide releasing compound.

The dressing body may be further impregnated with an additional antimicrobial agent. Non-limiting examples of the additional antimicrobial agent include chlorhexidine diacetate, chlorhexidine base, chlorhexidine gluconate, and mixtures thereof. Further non-limiting examples of the additional antimicrobial agent include silver, silver-sulfadiazine, and mixtures thereof. Other non-limiting examples of the additional antimicrobial agent include ethyl violet, gentian violet, methylene blue, and mixtures thereof. The additional antimicrobial agent may be impregnated into the dressing body by exposing the dressing body to a solvent having the additional antimicrobial agent dissolved therein. The additional antimicrobial agent may be impregnated into the dressing body using the same solvent system as the catalyst and/or nitric oxide releasing compound, discussed above, either during the same impregnation step, a subsequent impregnation step, or a prior impregnation step. The dressing body is exposed to the solvent solution for sufficient time to permit the additional antimicrobial agent to penetrate the dressing body. The impregnating step may occur at room temperature. The impregnating step may occur at a temperature in the range from about 25 to 55° C. Any solvent that is compatible with the additional antimicrobial agent and dressing body may be used, including those described above in relation to the nitric oxide releasing compound.

The dressing body may be fabricated of any physiologically compatible material that is capable of being impregnated with the nitric oxide releasing compound and releasing nitric oxide. The dressing body material also functions as a medical device insertion site dressing. The dressing body material is also capable of being impregnated with the catalyst and additional antimicrobial agent. Non-limiting examples of suitable dressing body materials include oxidized cellulose foam, collagen fibrils, and alginate hydrogel.

The dressing body may take any geometric shape. In one preferred embodiment, the dressing body is substantially disk-shaped. Other non-limiting geometric shapes for the dressing body include oval, triangle, square, rectangle, pentagon, hexagon, octagon, etc. The dressing body may comprise a central aperture for reception of the medical device. The central aperture may have a diameter in the range of 0.04 inches to 0.3 inches. The dressing body may have an outer dimension or diameter in the range of 0.5 inches to 3 inches. The dressing body may have a thickness in the range of 0.03 inches to 0.2 inches.

The self-activating dressing is particularly configured for use with a medical device inserted into a skin surface of a patient via a skin insertion site. The medical device may be a catheter.

Various embodiments are listed below. It will be understood that the embodiments listed below may be combined not only as listed below, but in other suitable combinations in accordance with the scope of the invention.

In an aspect, a self-activating dressing for use with a medical device inserted into a skin surface of a patient via a skin insertion site, comprising: a dressing body impregnated with a nitric oxide releasing compound which reacts in the presence of a physiological fluid to release nitric oxide; and a slit defined in the body configured to enable the body to be placed around a perimeter of the medical device on the skin surface at the skin insertion site such that the dressing body surrounds and contacts skin insertion site.

In one or more embodiments, the nitric oxide releasing compound impregnated in the dressing body may be selected from s-nitroso-n-acetyl penicillamine (SNAP), s-nitrosoglutathione (GSNO), and mixtures thereof.

In one or more embodiments, the physiological fluid may be selected from sweat, interstitial fluid, and blood.

In any embodiment herein, the dressing body may be further impregnated with a catalyst to facilitate release of nitric oxide. The catalyst may be selected from copper, iron, zinc, selenium, and silver.

In any embodiment herein, the dressing body may be further impregnated with an additional antimicrobial agent. The additional antimicrobial agent may be selected from chlorhexidine diacetate, chlorhexidine base, chlorhexidine gluconate, and mixtures thereof. The additional antimicrobial agent is selected from silver, silver-sulfadiazine, and mixtures thereof. The additional antimicrobial agent may be selected from ethyl violet, gentian violet, methylene blue, and mixtures thereof.

In any embodiment herein, the dressing body may comprise oxidized cellulose foam. In any embodiment herein, the dressing body may comprise collagen fibrils. In any embodiment herein, the dressing body may comprise alginate hydrogel.

In any embodiment herein, the dressing body may be substantially disk-shaped. In any embodiment herein, the dressing body may comprise a central aperture for reception of the medical device. The central aperture may have a diameter in the range of 0.04 inches to 0.3 inches. In any embodiment herein, the dressing body may comprise a slit extending from the central aperture to an outer perimeter of the dressing body.

In any embodiment herein, the dressing body may have an outer diameter in the range of 0.5 inches to 3 inches. In any embodiment herein, the dressing body may have a thickness in the range of 0.03 inches to 0.2 inches.

In any embodiment herein, the medical device may be a percutaneous device such as a catheter.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1 is a perspective view of an insertion site dressing, according to some embodiments;

FIG. 2 is an enlarged perspective view of an insertion site dressing, according to some embodiments; and

FIG. 3 is a perspective view of an insertion site dressing positioned about a medical device on a skin surface of a patient, according to some embodiments.

### DESCRIPTION OF EMBODIMENTS

The disclosure relates to a self-activating antimicrobial catheter insertion site dressing impregnated with a nitric oxide (NO) releasing compound. The nitric oxide releasing compound is activated in the presence of physiological fluids to release nitric oxide. Nitric oxide exhibits strong broad-spectrum antimicrobial properties and promotes wound healing at the insertion site.

Nitric oxide is a natural antimicrobial agent. Nitric oxide reacts with physiological concentrations of superoxide to create peroxynitrite which induces oxidative stress, nitrosates amino acids of bacterial cells, oxidizes and breaks their DNA strands, and causes cell membrane damage via lipid peroxidation. Additionally, nitric oxide reacts with oxidators to form N₂O₃ which reacts with sulfhydryl groups of cysteine residues on bacteria membrane proteins and alters or inhibits their functionality.

By incorporating a nitric oxide releasing compound into the insertion site dressing, the nitric oxide releasing compound will react and degrade in the presence of a physiological fluid, such as sweat, interstitial fluid, or blood, and release nitric oxide in the gaseous phase at physiologically relevant levels to exert the above described physiological mechanisms at the insertion site. In addition, nitric oxide possesses synergistic properties with common antimicrobial agents, like chlorhexidine or silver, for enhanced functionality.

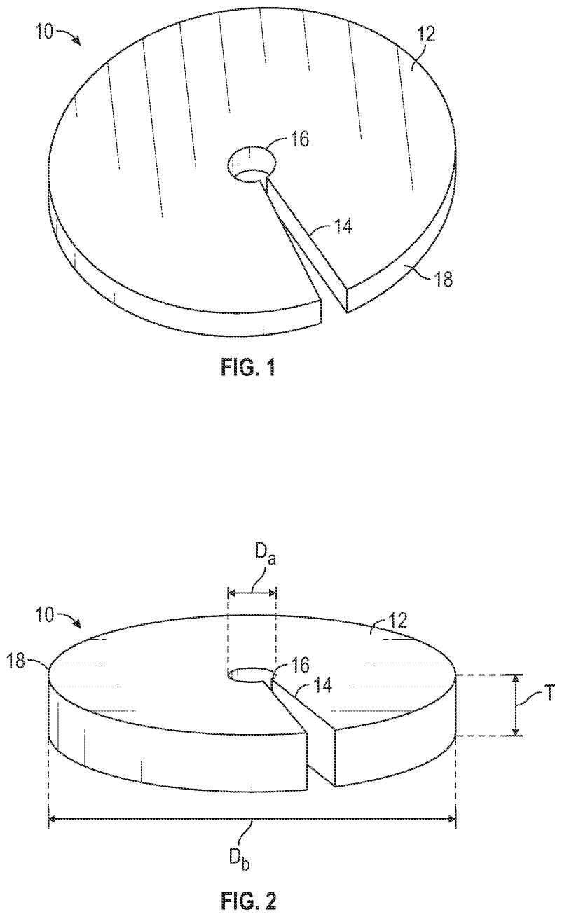

Reference is made to FIGS. 1 and 2 which illustrate a self-activating antimicrobial insertion site dressing 10 for use with a medical device inserted into a skin surface of a patient via a skin insertion site. The dressing 10 includes a dressing body 12 impregnated with a nitric oxide releasing compound which reacts in the presence of a physiological fluid to release nitric oxide.

The dressing body 12 described herein may be of any suitable shape. In the embodiment shown in FIGS. 1 and 2, the dressing body 12 has a circular or disk shape. Other suitable shapes include, but are not limited to oval, triangle, square, rectangular, hexagonal, octagonal, or any polygonal shape. One skilled in the art would understand how to modify the shape and size, including the length, width, and/or diameter, of the devices of the present disclosure based on one's anticipated outcome, including but not limited to, intended use of the device and intended dosage and release profile of a nitric oxide releasing compound and any other antimicrobial or biologically active agents.

One or more nitric oxide releasing compounds are integrated into the dressing 10, which releases nitric oxide in the presence of a physiological fluid. Any physiologically compatible nitric oxide releasing compound may be used herein. Non-limiting examples of nitric oxide releasing compounds include s-nitroso-n-acetylpenicillamine (SNAP), s-nitrosoglutathione (GSNO), and mixtures thereof.

The nitric oxide releasing compound may be impregnated in the dressing body 12 by exposing the dressing body 12 to a solvent having the nitric oxide releasing compound dissolved therein. The dressing body 12 is exposed to the solvent solution for sufficient time to permit the nitric oxide releasing compound to penetrate the dressing body 12. The impregnating step may occur at any suitable temperature. The impregnating step may occur at room temperature. The impregnating step may occur at a temperature in the range from about 25 to 55° C. Any solvent that is compatible with the dressing body and the nitric oxide releasing compound may be used.

The nitric oxide releasing compound may be dissolved in tetrahydrofuran (THF), dioxolane, methyl ethyl ketone (MEK), methanol, ethanol, isopropyl alcohol, water, or combinations thereof. The dressing may be soaked in these solutions containing the nitric oxide releasing compound for sufficient time to impregnate the dressing with the nitric oxide releasing compound. The exposure time may range between 5 minutes and 24 hours.

The dressing body 12 may be further impregnated with a catalyst to facilitate release of nitric oxide. Non-limiting examples of such catalysts include copper, iron, zinc, selenium, and silver. The catalyst may be impregnated into the dressing body 12 by exposing the dressing body to a solvent having the catalyst dissolved therein. The catalyst may be impregnated into the dressing body using the same solvent system as the nitric oxide releasing compound, discussed above, either during the same impregnation step, a subsequent impregnation step, or a prior impregnation step. The dressing body is exposed to the solvent solution for sufficient time to permit the catalyst to penetrate the dressing body. The impregnating step may occur at any suitable temperature. The impregnating step may occur at room temperature. The impregnating step may occur at a temperature in the range from about 25 to 55° C. Any solvent that is compatible with the dressing body and the catalyst may be used.

The dressing body may be further impregnated with an additional antimicrobial agent. Non-limiting examples of the additional antimicrobial agent include chlorhexidine diacetate, chlorhexidine base, chlorhexidine gluconate, and mixtures thereof. Additional non-limiting examples of the additional antimicrobial agent include silver, silver-sulfadiazine, and mixtures thereof. Other non-limiting examples of the additional antimicrobial agent include ethyl violet, gentian violet, methylene blue, and mixtures thereof. The additional antimicrobial agent may be impregnated into the dressing body by exposing the dressing body to a solvent having the additional antimicrobial agent dissolved therein. The additional antimicrobial agent may be impregnated into the dressing body using the same solvent system as the catalyst and/or nitric oxide releasing compound, discussed above, either during the same impregnation step, a subsequent impregnation step, or a prior impregnation step. The dressing body is exposed to the solvent solution for sufficient time to permit the additional antimicrobial agent to penetrate the dressing body. The impregnating step may occur at any suitable temperature. The impregnating step may occur at room temperature. The impregnating step may occur at a temperature in the range from about 25 to 55° C. Any solvent that is compatible with the dressing body and the additional antimicrobial agent may be used.

The dressing body 12 may be fabricated of any physiologically compatible material that is capable of being impregnated with the nitric oxide releasing compound and releasing nitric oxide. Non-limiting examples of suitable dressing body materials include oxidized cellulose foam, collagen fibrils, and alginate hydrogel.

The dressing 10 described herein is configured for use with a percutaneous medical device, such as an indwelling catheter, that has punctured the skin of a patient and has a portion of the catheter protruding from the skin. The dressing body includes a slit 14 configured to enable the dressing body to be placed around a perimeter of the medical device on the skin surface at the skin insertion site such that the dressing body surrounds and contacts skin insertion site. The slit 14 can be formed in the dressing body 12 by cutting, punching, or other similar mechanical forming technique. The width of slit 14 is adapted to facilitate installation over the already installed percutaneous medical device. The width of slit may range from very small when the sides of the slit touch each other (i.e. a cut with a very narrow blade), corresponding to a slit from about less than 0.004 inches gap to about 0.04 inches gap. The slit 14 enables the dressing to fully surround the percutaneous medical device at the insertion or puncture site.

The dressing body 12 may take any geometric shape. In one preferred embodiment, the dressing body is substantially disk-shaped. Other non-limiting geometric shapes for the dressing body include oval, triangle, square, rectangle, pentagon, hexagon, octagon, etc.

The dressing body 12 may include a central aperture 16 for reception of the medical device. The size or diameter (Dₐ) of the central aperture 16 is adapted for fully surrounding the medical device protruding from the insertion site in a snug or loose configuration, with the size or diameter (Dₐ) of the aperture typically ranging from about 90 percent of the outside diameter of the medical device to about 150 percent of the outside diameter of the medical device. The central aperture may have a size or diameter (Dₐ) in the range of 0.04 inches to 0.3 inches.

The slit 14 extends from the central aperture 16 to an outer perimeter 18 of the dressing body.

The dressing body may have an outer size or diameter (Db) in the range of 0.5 inches to 3 inches.

The thickness (T) of the dressing body 12 may be varied as desired, depending upon the desired pharmaceutical dosage of nitric oxide, and any other antimicrobial or biologically active agents impregnated in the dressing body, and duration of delivery. A suitable pad thickness will be in a range of about 0.03 inches to 0.2 inches.

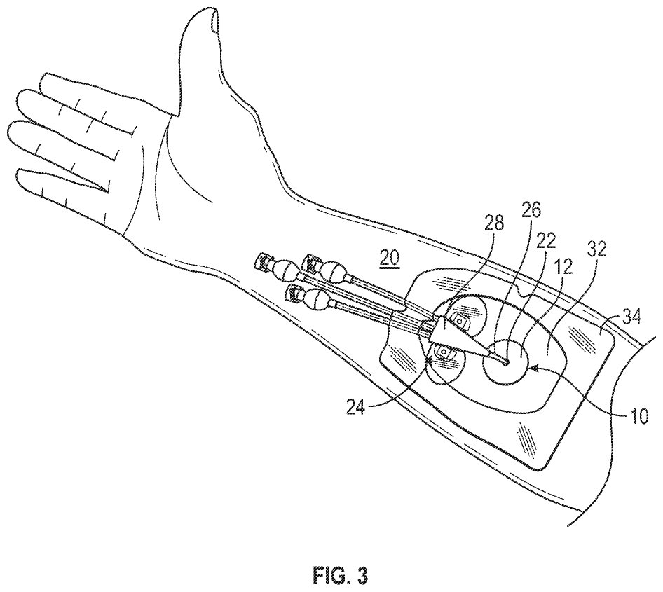

FIG. 3 is a perspective view of one exemplary use of the dressing 10. positioned about a medical device on a skin surface 20 of a patient. The dressing 10 includes a dressing body 12 that covers a skin insertion site 22 through which a medical device, such as a catheter assembly 24, passes for disposal within the body of a patient.

As shown in FIG. 3, the catheter assembly 24, includes a catheter tube 26 and a hub 28 attached to a proximal end of the catheter tube 26. The catheter tube 26 extends through the skin surface into the patient via the skin insertion site 22.

Though the discussion herein focuses on use of the dressing with a peripheral IV-type of catheter, other types of catheters and medical devices can benefit from use of the dressing. Non-limiting examples of such catheters and medical devices include central venous catheters, peripheral venous catheters, or any other indwelling catheters for delivery into and/or sampling from the patient. All of these indwelling catheters, when in place, have a portion of the catheter device that is external and left protruding from the skin, which can be the cause of infection around the insertion sites of the medical devices.

An adhesive (not shown) may optionally be provided on a bottom surface of the dressing body 12, which is configured to adhere the dressing 10 to the skin surface of the patient.

The slit 14 enables the dressing body 12 to fully surround and contact the skin insertion site 22, through which the catheter tube 26 passes, about the perimeter of the catheter tube (or other medical device passing through the skin). This leaves no portion of the region immediately surrounding the skin insertion site 22 uncovered. In response to a physiologic fluid, such as sweat, interstitial fluid, or blood, the nitric oxide releasing compound within the dressing body releases nitric oxide to contact the skin surface at the skin insertion site 22. In this manner, the dressing is self-activating in response to a physiologic fluid. The released nitric oxide assists in preventing the colonization of microbes and promotes wound healing.

A dressing film 32 may optionally be provided with an inner surface facing the patient's skin and an outer surface facing away from the patient's skin. The dressing film 32 can be formed from any physiologically compatible adhesive translucent or transparent dressing for wounds, such as polyurethane film or copolyester film. The film may have a thickness of about 50 to 350 microns, preferably 100-200 microns. Other suitable materials for the dressing film 32 include transparent polyester films with pressure sensitive biocompatible adhesive. A pressure sensitive adhesive may be disposed on the inner surface of the dressing film 32. The pressure-sensitive adhesive can be any pressure sensitive adhesive known in the art. The adhesive can be continuous or discontinuous, i.e. applied in a patterned fashion. In one embodiment, the adhesive is applied in stripes, thus providing for breathability of the dressing. In another embodiment, the adhesive is applied to a perimeter frame 34 of the dressing film and not to the dressing film surrounded by the perimeter frame 34, thereby creating in the area over the dressing 10 and catheter assembly 24 without adhesive, which may facilitate removal of the dressing 10 during dressing change.

In one embodiment, the dressing film 32 is at least partially translucent or transparent to allow a healthcare professional to visually check on the dressing 10 and catheter assembly 24.

The disclosed dressing 10 which released nitric oxide at the medical device insertion site provides a hemostatic and wound healing activity. The dressing may control minor bleeding at the percutaneous medical device insertion access site. Moreover, the present disclosure promotes wound healing while providing protection at the insertion site by slow release of nitric oxide, a broad spectrum antimicrobial agent to help resist microbial colonization of the dressing.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present inventions have been described in detail, it should be understood that the various changes, substitutions, and alterations could be made hereto without departing from the spirit and scope of the invention.

1. A self-activating dressing for use with a medical device inserted into a skin surface of a patient via a skin insertion site, comprising:
a dressing body impregnated with a nitric oxide releasing compound which reacts in the presence of a physiological fluid to release nitric oxide; and
a slit defined in the dressing body configured to enable the dressing body to be placed around a perimeter of the medical device on the skin surface at the skin insertion site such that the dressing body surrounds and contacts skin insertion site.

2. The self-activating dressing of claim 1, wherein the nitric oxide releasing compound impregnated in the dressing body is selected from s-nitroso-n-acetyl penicillamine (SNAP), s-nitrosoglutathione (GSNO), and mixtures thereof.

3. The self-activating dressing of claim 1, wherein the physiological fluid is selected from sweat, interstitial fluid, and blood.

4. The self-activating dressing of claim 1, wherein the dressing body is further impregnated with a catalyst to facilitate release of nitric oxide.

5. The self-activating dressing of claim 4, wherein the catalyst is selected from copper, iron, zinc, selenium, and silver.

6. The self-activating dressing of claim 1, wherein the dressing body is further impregnated with an additional antimicrobial agent.

7. The self-activating dressing of claim 6, wherein the additional antimicrobial agent is selected from chlorhexidine diacetate, chlorhexidine base, chlorhexidine gluconate, and mixtures thereof.

8. The self-activating dressing of claim 6, wherein the additional antimicrobial agent is selected from silver, silver-sulfadiazine, and mixtures thereof.

9. The self-activating dressing of claim 6, wherein the additional antimicrobial agent is selected from ethyl violet, gentian violet, methylene blue, and mixtures thereof.

10. The self-activating dressing of claim 1, wherein the dressing body comprises oxidized cellulose foam.

11. The self-activating dressing of claim 1, wherein the dressing body comprises collagen fibrils.

12. The self-activating dressing of claim 1, wherein the dressing body comprises alginate hydrogel.

13. The self-activating dressing of claim 1, wherein the dressing body is substantially disk-shaped.

14. The self-activating dressing of claim 1, wherein the dressing body comprises a central aperture for reception of the medical device.

15. The self-activating dressing of claim 14, wherein the central aperture has a diameter in the range of 0.04 inches to 0.3 inches.

16. The self-activating dressing of claim 1, wherein the dressing body has an outer diameter in the range of 0.5 inches to 3 inches.

17. The self-activating dressing of claim 1, wherein the dressing body has a thickness in the range of 0.03 inches to 0.2 inches.

18. The self-activating dressing of claim 13, wherein the dressing body comprises a central aperture for reception of the medical device, wherein the central aperture has a diameter in the range of 0.04 inches to 0.3 inches, wherein the dressing body has an outer diameter in the range of 0.5 inches to 3 inches, and wherein the dressing body has a thickness in the range of 0.03 inches to 0.2 inches.

19. The self-activating dressing of claim 1, wherein the medical device is a catheter.

20. The self-activating dressing of claim 1, wherein the dressing body comprises oxidized cellulose foam, wherein the nitric oxide releasing compound impregnated in the dressing body is s-nitroso-n-acetyl penicillamine (SNAP), wherein the dressing body is further impregnated with silver sulfadiazine as an additional antimicrobial agent, wherein the dressing body is further impregnated with a silver catalyst to facilitate release of nitric oxide.
